(19) United States
(12) Patent Application Publication
(10) Pub. No.: US 2004/0003679 A1
(43) Pub. Date: Jan. 8, 2004

(54) Apparatus and method for in vitro recording and stimulation of cells

(76) Inventors: David Ide, Arlington, VA (US); George Z. Mentis, Bethesda, MD (US)

Correspondence Address: MERCHANT & GOULD PC, P.O. BOX 2903, MINNEAPOLIS, MN, 55402-0903, US

(21) Appl. No.: 10/190,215

(22) Filed: Jul. 5, 2002

(51) Int. Cl. C12M 1/34

(52) U.S. Cl. 74/813 C; 435/287.2

(57) ABSTRACT

The invention includes a device having a chamber, at least one tool, and at least one micromanipulator that has a ball housing having a space therein, a ball assembly, having a ball with a hole and a tube, and a chamber attachment, wherein the ball assembly is movably positioned within the space in the ball housing, and the tube is securely positioned within the hole in the ball, wherein the ball imparts three dimensional movement to the tube through rotational movement of the ball within the ball housing, wherein the micromanipulator is reversibly attached to the chamber and the micromanipulator functions to manipulate the at least one tool with respect to the sample in the chamber.

### FIELD OF THE INVENTION

The invention relates generally to devices for the manipulation of electrical equipment used for the stimulation and recording of neural impulses. More specifically, the invention relates to devices and methods allowing micromanipulation of electrodes in very small spaces across multiple sites simultaneously.

### BACKGROUND OF THE INVENTION

There continues to be a demand for experiments performed in vitro, where the tissue under investigation is kept alive under artificial but physiologically normal conditions. Such experimental conditions usually require absolute control of the specimen through control of environmental conditions as well as physical control and manipulation of the sample, the control of tools, and the application of chemicals and or drugs.

Manipulation of the tools used in an experiment may be important in many different respects. Controlled movement may be important to allow access to different areas or sites on the sample. At the same time, fixed positioning of the tools can also be important to allow for secure sampling, which provides experimentation of high integrity. Examination, such as recording of electrical signals, across many sites may also be relevant in certain specimens such as neural tissue.

Examples of previous apparatus used for manipulation of samples include Narishige et al., U.S. Pat. Nos. 4,529,169 and 4,679,976 which disclose a system for manipulation of any number of devices including glass electrodes with the aid of a hydraulic remote control apparatus and X, Y, and Z coordinate actuators.

Yoneyanra, U.S. Pat. No. 5,771,749 discloses a slide mechanism with rough and fine adjustment settings. Further, U.S. Pat. No. 5,845,541 also discloses a micromanipulator operated and controlled with a computer mouse. The disclosed system is intended to provide fine control over complicated operations and procedures.

Additionally, U.S. Pat. No. 4,901,446 discloses a vertical device for actuating glass electrodes in the X- and Y-coordinates. The device has spring actuated lever rods to manipulate samples to various coordinates. The device is apparently intended to provide fine manipulation of tools used to work with cells and cell membranes.

The disclosed devices provide relevant examples of micromanipulator devices. However, there remains a need for micromanipulating devices used with any number of tools in very small spaces across multiple sites simultaneously.

### SUMMARY OF THE INVENTION

One embodiment of the invention includes a micromanipulator with a ball housing having a space therein, and a ball assembly, having a ball with a hole therein, and a tube, wherein the ball assembly is movably positioned within the space in the ball housing, and the tube is securely positioned within the hole in the ball, wherein the ball imparts three dimensional movement to the tube through rotational movement of the ball within the ball housing.

Another embodiment of the invention includes a device having a chamber, at least one tool, and at least one micromanipulator that has a ball housing having a space therein, a ball assembly, having a ball with a hole and a tube, and a chamber attachment, wherein the ball assembly is movably positioned within the space in the ball housing, and the tube is securely positioned within the hole in the ball, wherein the ball imparts three dimensional movement to the tube through rotational movement of the ball within the ball housing, wherein the micromanipulator is reversibly attached to the chamber and the micromanipulator functions to manipulate the at least one tool with respect to the sample in the chamber.

### BRIEF DESCRIPTION OF THE DRAWINGS

FIG. 1 is a depiction of a device in accordance with one aspect of the invention.

FIG. 2 is a depiction of a device in accordance with another aspect of the invention.

FIG. 3A is a front view of a micromanipulator in accordance with one aspect of the invention.

FIG. 3B is a perspective view of a micromanipulator in accordance with one aspect of the invention.

FIG. 3C is a perspective view of a micromanipulator in accordance with another aspect of the invention.

FIG. 3D is a perspective view of a micromanipulator in accordance with yet another aspect of the invention.

FIG. 4 is a top view of a device in accordance with one aspect of the invention.

FIG. 5A is a top view of a chamber in accordance with one aspect of the invention.

FIG. 5B is a top view of a chamber in accordance with another aspect of the invention.

FIG. 6A is a perspective view of a device in accordance with one aspect of the invention.

FIG. 6B is a perspective view of a device in accordance with another aspect of the invention.

FIG. 6C is a perspective view of a device in accordance with yet another aspect of the invention.

FIG. 7 is a perspective view of a base and chamber in accordance with one aspect of the invention.

FIG. 8 is a more detailed view of the chamber of FIG. 7.

FIG. 9 is a perspective view of a tissue sample with a number of extracellular electrodes in accordance with the invention configured to analyze the tissue sample.

FIG. 10 depicts traces of extracellular recordings that were recorded using a device in accordance with one aspect of the invention.

FIG. 11 shows a close up view (magnification: 10×) of an epifluorescent microscope configured with a device in accordance with one aspect of the invention.

FIGS. 12A and 12B show current to voltage relationships from a motorneuron obtained using a device in accordance with the invention. FIG. 12A shows the current injected and FIG. 12B shows the resulting changes in the membrane potential of the motorneuron.

### DETAILED DESCRIPTION OF THE PREFERRED EMBODIMENTS

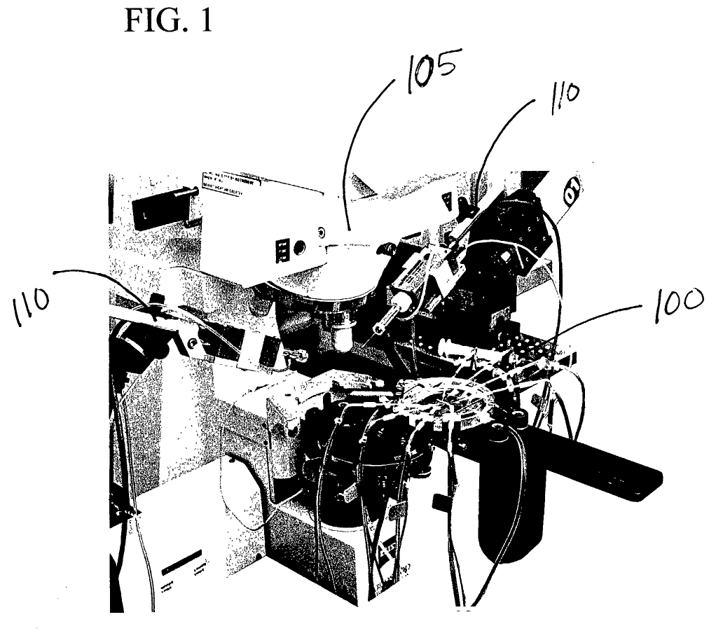

FIG. 1 depicts a device in accordance with one aspect of the invention in an exemplary configuration within a system for obtaining measurements and/or setting the system up for obtaining measurements. This figure shows the device 100, a microscope 105, and intracellular electrodes 110. The device 100 functions to hold the specimen under examination and manipulate and/or position analysis tools in proximity to the specimen. The microscope 105 in this depiction can be used for placing the intracellular electrodes 110. The intracellular electrodes 110 are used to provide electrical stimulation and/or measure electrical signals from cells or neurons. It should be understood that numerous types and configurations of microscopes 105, intracellular electrodes 110, various other equipment, and analysis tools can be utilized along with a device 100 of the invention and still be within the scope of the invention. FIG. 1 represents only one configuration of a device 100 with a microscope 105 and two intracellular electrodes 110.

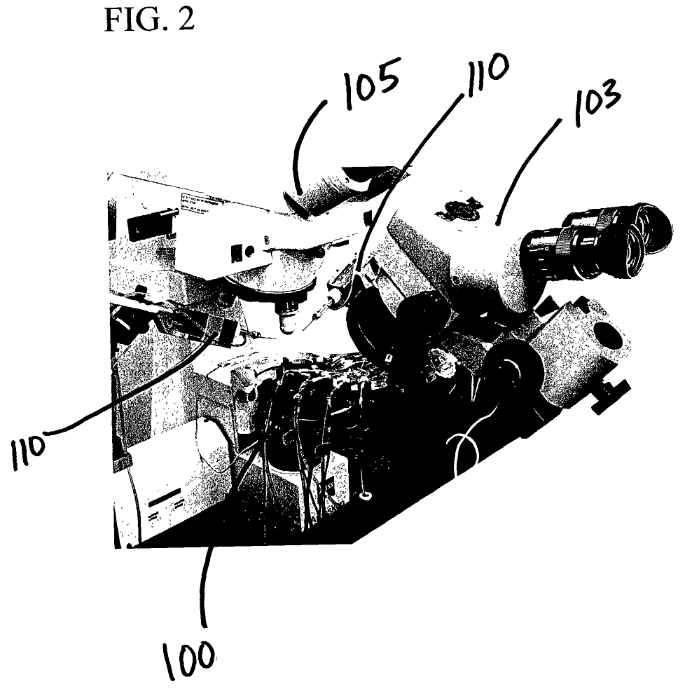

FIG. 2 depicts a device in accordance with one aspect of the invention in another exemplary configuration within a system for obtaining measurements and/or setting the system up for obtaining measurements. This figure shows the device 100, a pivot-dissecting microscope 103, a standard microscope 105, and two intracellular electrodes 110. The device 100 functions to hold the specimen and manipulate and/or position the analysis tool relative to the specimen. The pivot-dissecting microscope 103 can be used to position the analysis tools. The intracellular electrodes 110 can be used to provide electrical stimulation and/or measure electrical signals from within intracellular spaces. It should be understood that numerous types and configurations of microscope 105, pivot-dissecting microscope 103, intracellular electrodes 110 and other equipment can be used in a system along with a device 100 of the invention and still be within the scope of the invention. FIG. 2 represents only one exemplary configuration of a system that includes a device 100 of the invention with a standard microscope 105, a pivot-dissecting microscope 103, and two intracellular electrodes 110.

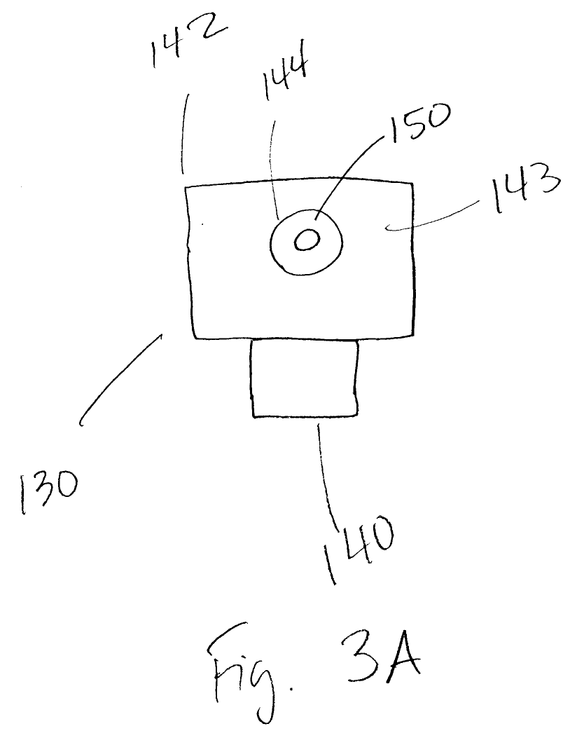

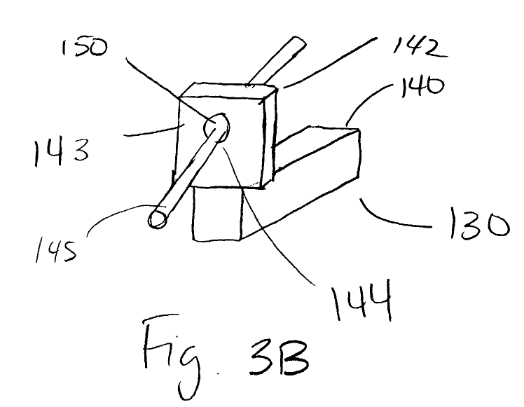

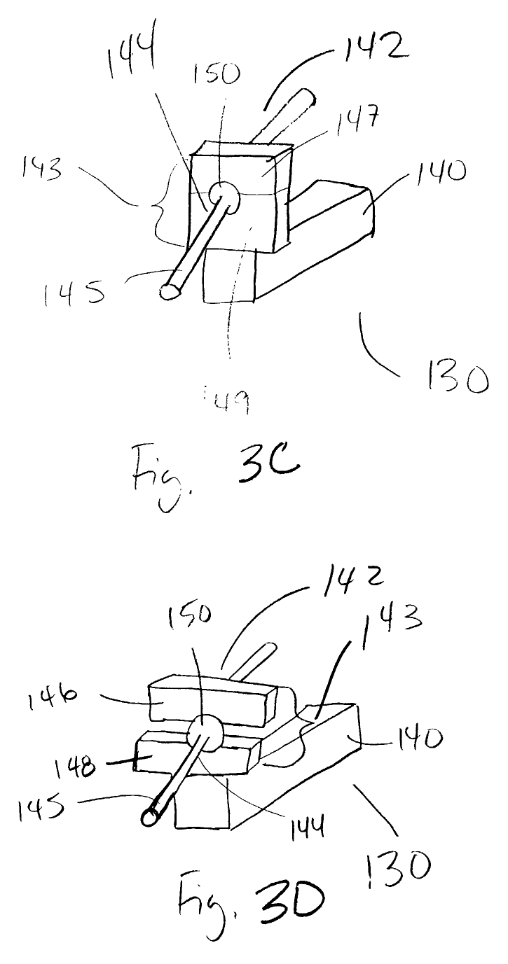

A device 100 in accordance with the invention includes at least one micro-manipulator 130, an example of which can be seen in FIG. 3A. The micro-manipulator 130 functions to allow the user to interact with the specimen. More specifically, the micro-manipulator 130 functions to bring an analysis tool in close proximity with the specimen. Examples of analysis tools can include, but are not limited to, microelectrodes, suction tools (for suction of solution for example), temperature probes, and holders for multi-barrel probes that can be used to apply chemicals or drugs to local areas of the specimen. The micro-manipulators 130 also function to allow intricate control of the analysis tools with respect to the specimen. For example, in an embodiment of the invention where the analysis tool is a microelectrode, the micro-manipulator 130 can allow four dimensional manipulation of the microelectrode with respect to the specimen.

Referring again to FIG. 3A, the micro-manipulator 130 includes a motion assembly 142. Motion assembly 142 includes ball housing 143, and ball assembly 144. Motion assembly 142 functions to provide three dimensional movement of the analysis tool that it utilizes. In one embodiment, this movement is accomplished by the configuration of the components of the motion assembly 142. The ball assembly 144 includes a ball 150. The ball 150 has a hole through the entirety of it. In this embodiment, the analysis tool is placed through the hole in the ball 150 so that the micromanipulator 130 can effect movement and manipulation thereof.

In another embodiment, seen in FIG. 3B, the ball assembly 144 can further include a tube 145. The tube 145, which, in this embodiment, is securely positioned within a hole that has been formed through the entirety of the center of the ball 150. The ball assembly 144 is then movably positioned within a space in the ball housing 143. The ball assembly 144 is positioned inside the ball housing 143 so that it can move within the space, but not be removed from within the space without a positive action by the user. This allows the ball 150 to impart three dimensional movement to the tube 145 through rotational movement of the ball 150 within the ball housing 143.

In one embodiment, the ball assembly 144 can be formed as one continuous component. In another embodiment, the ball 150 and the tube 145 can be formed separately and the tube 145 inserted through a hole formed in the ball 150. The tube 145 can also alternatively be sealed within the hole in the ball 150. Sealing the tube 145 within the ball 150, if undertaken, can be accomplished through the use of adhesive, soldering, press fit, set screw, or other methods known to those of skill in the art having read this specification. In one embodiment, the tube 145 is sealed within the ball 150 by soldering.

The ball 150 is generally spherical in shape. In one embodiment, the ball 150 is a sphere. The dimensions of the ball 150 will depend on the dimensions of the ball housing 143 and the dimensions of the tube 145. In an embodiment where the micro-manipulator 130 is to be used for the manipulation of microelectrodes, generally the ball 150 has a radius of from about 3 mm to about 10 mm. In another embodiment, ball 150 has a radius of about 3 mm to about 7 mm.

The ball 150 can be made of any material that allows the ball assembly 144 to provide three dimensional motion. Examples of materials that can be used to fabricate the ball 150 include, but are not limited to, brass, stainless steel, and plastic. In one embodiment, the ball 150 is made of brass.

The tube 145 is generally a hollow cylinder. In one embodiment, the tube 145 is a hollow cylinder with a length that is larger than its radius. The specific dimensions of the tube 145 will depend on the dimensions of the ball 150 and any tool that may be housed within tube 145. Generally, the larger the tool to be housed within the tube 145, the larger the tube 145 must be and therefore, the larger the ball 150 must be. In an embodiment where the micro-manipulator 130 is to be used for the manipulation of microelectrodes, generally the tube 145 has a radius of about 1 mm to about 2 mm, and a length of about 1 cm to about 5 cm. In another embodiment, tube 145 has a radius of about 2 mm, and a length of about 4 cm.

The tube 145 can be made of any material which allows the ball assembly 144 to function to provide three dimensional motion and house any tool that may be used therewith. Examples of materials that can be used to fabricate the tube 145 include, but are not limited to, brass, stainless steel, plastic, aluminum, and titanium. In one embodiment, the tube 145 is made of stainless steel.

Referring again to FIG. 3B, the ball housing 143 functions to house the ball assembly 144 within the micro-manipulator 130. The ball housing 143 may either be formed around or be put in place around the ball assembly 144. The ball housing 143 is generally configured to maintain the position of the ball assembly 144 within the ball housing 143 but allow movement of the ball assembly 144 within the space in the ball housing 143.

In one embodiment, the ball housing 143 includes one continuous component. In another embodiment, the ball housing 143 includes more than one component. In such an embodiment, two or more parts can be fabricated, positioned around the ball assembly 144 and secured in place. Examples of such embodiments are depicted in FIGS. 3C and 3D. In the embodiment depicted in FIG. 3C, the ball housing 143 includes an upper portion 147 and a lower portion 149 that are configured around ball assembly 144 and are in physical contact with each other. In the embodiment depicted in FIG. 3D the ball housing 143 includes an upper portion 146 and a lower portion 148 that are configured around ball assembly 144 but are not in physical contact with each other. In one embodiment, the upper portion 146 and the lower portion 148 are held tightly together by screws. The embodiment depicted in FIG. 3D can be used preferentially in a configuration of the device 100 where a lower profile is desired.

The dimensions of the ball housing 143 depend in part on the dimensions of the ball assembly 144. In an embodiment of the invention that can be used for microelectrode analysis of signals from the sample under investigation, where the sample can include a central nervous system slice, a spinal cord preparation, or a muscle preparation, the ball housing 143 has dimensions of about 1 cm×1 cm×0.3 cm.

The ball housing 143, whether one continuous component, or two or more components, can be made of any material which can function to movably house the ball assembly 144 within the ball housing 143. Examples of such materials include, but are not limited to, stainless steel, brass, Plexiglas®, titanium, aluminum, or plastic. In one embodiment, the ball housing 143 is made of an upper portion 147 or 146 and a lower portion 149 or 148, which are made of plastic, more specifically delrin plastic.

A micromanipulator 130 in accordance with the invention can also include an attachment assembly 140, as can be seen in FIGS. 3A, 3B, 3C, or 3D. The attachment assembly 140 functions to reversibly attach micro-manipulator 130 to the chamber 120 or the base 125. In one embodiment, attachment assembly 140 is configured to accept a screw which reversibly attaches the micro-manipulator 130 to the base 125. Attachment assembly 140 can be made of stainless steel, Plexiglas®, brass, aluminum, titanium, plastic, as well as similar materials known to those of skill in the art having read this specification. In one embodiment, attachment assembly 140 is made of anodized aluminum and is attached to the base 125 with at least one screw. In another embodiment, the attachment assembly 140 is configured to be attached to the chamber 120.

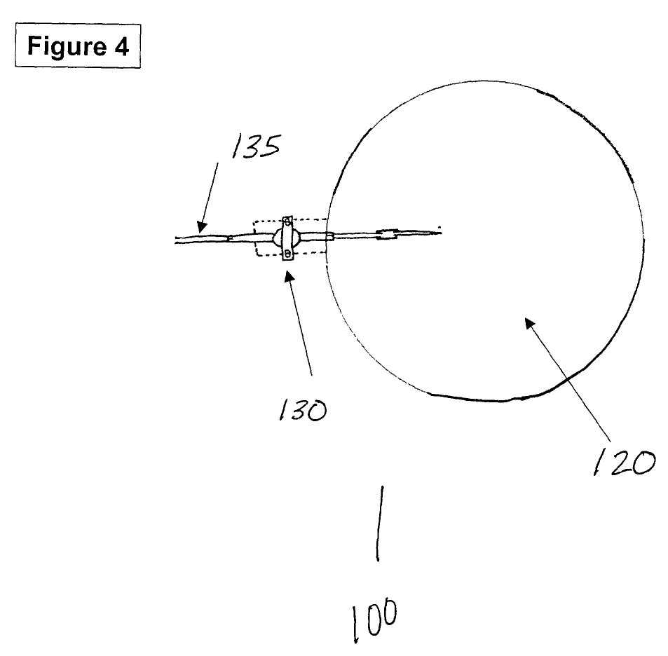

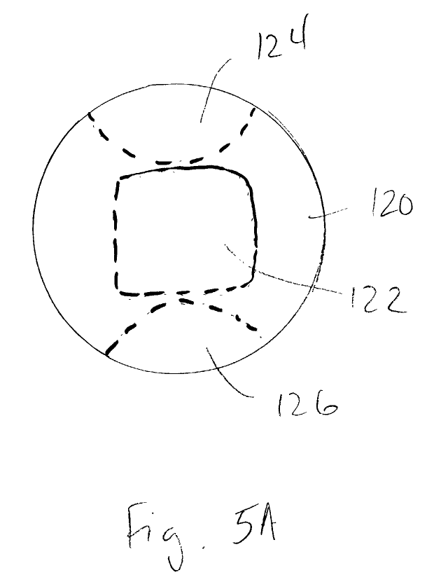

FIG. 4 depicts a device 100 of the invention in accordance with one aspect of the invention. The device 100 includes a chamber 120 and at least one micro-manipulator 130, which is optionally assembled with a glass electrode 135.

The chamber 120 functions to contain the specimen and allows at least one micro-manipulator 130 to interact with the specimen. The chamber 120 can have any configuration that allows the function thereof to be carried out. In one embodiment, the chamber 120 has a generally round configuration because it allows access of a number of micro-manipulators 130 from a number of different angles. The dimensions of chamber 120 can vary depending on the relative size of the specimen to be contained therein. Generally, larger chambers 120 allow for more micro-manipulators 130 to be configured around the chamber 120. In one embodiment of the invention where the device 100 is to be used for neurological testing of small mammals or portions of small mammals with glass electrode 135, the chamber has dimensions of about 5 cm to about 10 cm. The chamber 120 may be configured to receive at least one micro-manipulator 130.

Chamber 120 can be made of any material that can contain the specimen. In one embodiment, the chamber 120 can be made of a number of materials, including but not limited to Plexiglas®, stainless steel, plastic, titanium, and aluminum. In one embodiment, the chamber 120 is made of Plexiglas®.

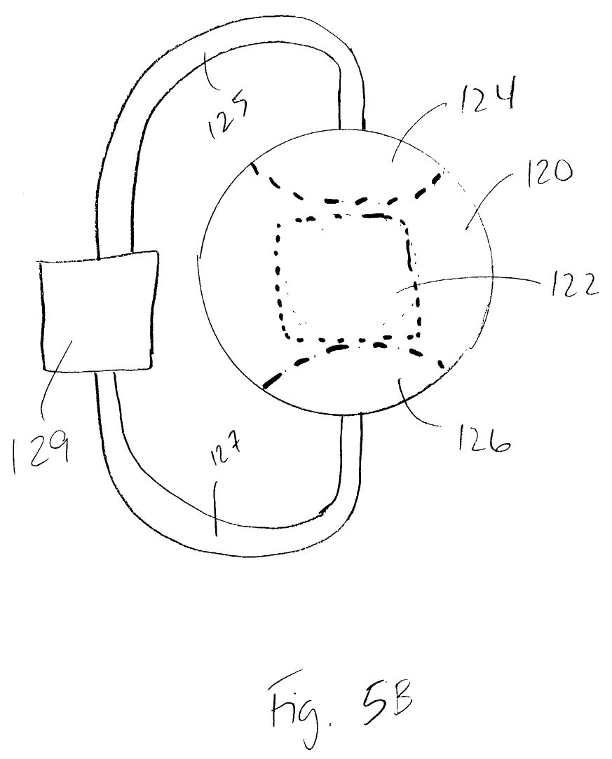

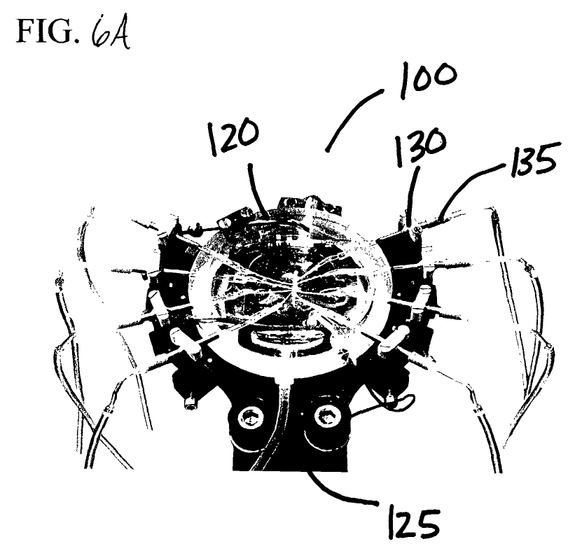

One embodiment of a device 100 in accordance with the invention, depicted in FIG. 5A has a chamber 120 that includes an inlet area 124, a specimen area 122, and an outlet area 126. A chamber 120 with such a configuration allows fluids to be circulated around the specimen. Circulation of fluids can be used for in vitro recording, to maintain the specimen in a certain condition, to prevent degradation, to mimic the surrounding area where the specimen originated from, or to perfuse the specimen with different drugs and/or chemicals. The inlet area 124 can be used to introduce the fluid. Generally, the inlet area 124 is a region of the overall chamber 120. In one embodiment, the inlet area 124 may be raised relative to the bottom surface of the chamber 120.

The specimen area 122 is the area where the specimen is actually placed within the chamber 120. Generally, the specimen area 122 is in the center of the chamber 120. In one embodiment, the bottom surface of the specimen area 122 may be covered with a material that does not allow the specimen to move or float freely in the fluid within the specimen area 122 and/or a material that allows the specimen to be easily pinned to the specimen area 122.

The outlet area 126 can be used to remove the fluid that is circulated. Generally, the outlet area 126 is a portion of the overall chamber 120. In one embodiment the outlet area 126 may be recessed relative to the bottom surface of the chamber 120.

FIG. 5B depicts a chamber 120 in accordance with another aspect of the invention. Chamber 120 includes an inlet area 124, a specimen area 122, and an outlet area 126 as described above, but it also contains an inlet tube 125, an outlet tube 127, and a fluid modulator 129. Inlet tube 125 functions to add fluid to the inlet area 124. Inlet tube 125 can be plastic, Tygon®, or polyvinyl chloride (PVC). In one embodiment, inlet tube 125 is made of Tygon®.

Outlet tube 127 functions to remove fluid from the outlet area 126. Outlet tube 127 can be plastic, Tygon®, or polyvinyl chloride (PVC). In one embodiment, outlet tube 127 is made of Tygon®.

Fluid modulator 129 functions to circulate the fluid through the inlet tube 125, the inlet area 124, the specimen area 122, the outlet area 126, the outlet tube 127, and through the fluid modulator 129 back into the inlet tube 125. Fluid modulator 129 may also function to maintain, or modify at least one property of the fluid. Examples of properties that may be maintained or modified include, but are not limited to, oxygen concentration, temperature, pH, ion concentration, and osmolarity. Alternatively, the fluid modulator 129 may be used to administer, monitor the concentration of, and/or regulate the concentration of drugs and/or chemicals. In one embodiment, fluid modulator 129 is positioned higher than the chamber 120 so that the flow of the fluid occurs at least partially because of gravity. In one embodiment, the fluid is removed from outlet area 126 and back to the fluid modulator 129 by means of a peristaltic pump.

In yet another embodiment, the inlet area 124 is connected to the tissue area 122, which is connected to the outlet area 126 by fine holes in the inlet area 124 and the outlet area 126. In this embodiment, fluid that is utilized in the chamber then flows from the inlet area 124 to the tissue area 122 and then on to the outlet area 126 by diffusion. In that manner, the tissue area 122 will not be affected by movement and/or vibration from ripples which can be caused by a relatively high flow rate of the solution in to the tissue area 122. This can be important for the stability and quality of specimen examination, especially during neurological experiments employing intracellular recordings.

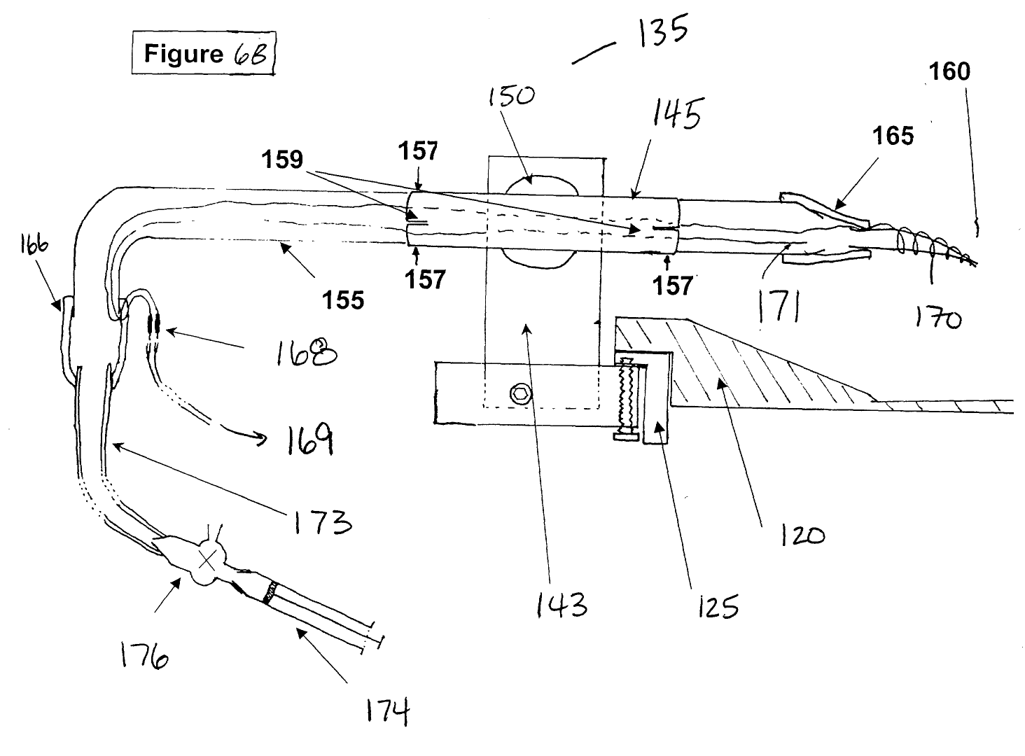

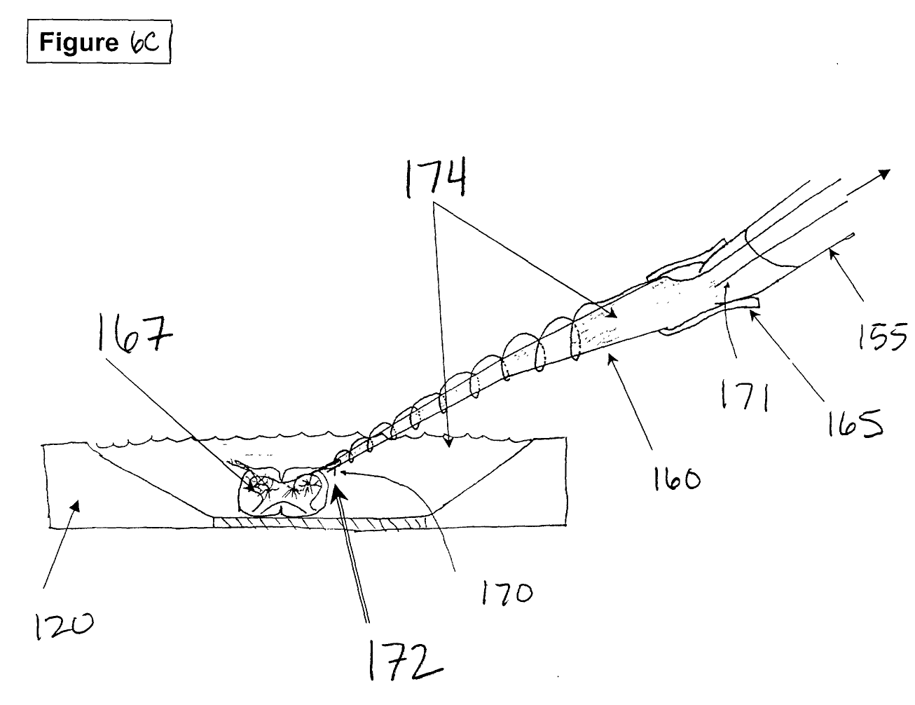

Another embodiment of a device 100 of the invention is depicted in FIG. 6A. This embodiment includes a chamber 120, a base 125, at least one micro-manipulator 130, and at least one glass electrode 135 that is used as an analysis tool. The chamber 120, and the at least one micro-manipulator 130 have similar functions and configurations as those that were discussed previously. The chamber 120 in this embodiment may be configured to be reversibly received by the base 125.

The base 125 functions to allow the device 100 to be securely positioned as part of a system for obtaining measurements. For example, the base 125 can function to allow the device 100 to be secured to an anti-vibration system that is often used for such measurements. In one embodiment of the invention, the base 125 can be configured to securely hold the chamber 120 and reversibly hold the at least one micromanipulator 130. Generally, the base 125 can be made from any material that allows the chamber to be securely integrated into the system. Examples, of such material include, but are not limited to stainless steel, aluminum, and titanium. In one embodiment, the base 125 is made of anodized aluminum.

The glass electrode 135, as depicted in FIG. 6B generally functions to provide stimulation to and/or record measurements from the specimen. A glass electrode 135 in accordance with the invention can include a glass tube 155 and a tip 160. The glass tube 155 is positioned inside the tube 145 of the ball assembly 144. The dimensions of the glass tube 155 depend at least in part on the dimensions of the micromanipulator 130. Generally, the glass tube 155 has an inside diameter of about 1 mm to about 3 mm. In one embodiment, the glass tube 155 has an inside diameter of about 1.4 mm. Generally, the glass tube 155 has an outside diameter of about 1.5 mm to about 4 mm. In one embodiment, the glass tube 155 has an outside diameter of about 2 mm.

The glass tube 155 is configured to so that is can be pushed or pulled into or out of the ball assembly 144 by the user by applying pressure, using his fingers, at the edges 157 of the ball assembly 144. This allows the tip 160 of the glass tube 155 to be controlled in a three dimensional area and effectively position the tip 160 at any point in a three dimensional space. In one embodiment, the movement of the glass tube 155 is made easier by a modification to the tube 145 of the ball assembly 144. In this embodiment, the tube 145 includes two slits 159, at the edges 157 of the tube 145. The inclusion of the slits 159 in the tube 145 allows the tube 145 to bend slightly in order to secure the glass electrode so that it does not rotate freely around its directional axis without a positive action by the user.

Generally, the glass electrode 135 also contains the tip 160, which is positioned at the end of the glass tube 155 closest to the sample to be investigated. In one embodiment, the tip 160 of the glass electrode 135 is generally made of polyethylene tubing with an inside diameter of about 0.6 mm and an outside diameter of about 1 mm. Another embodiment of the invention has the tip 160 of the glass electrode 135 made of polyethylene tubing with an inside diameter of about 0.58 mm and an outside diameter of about 0.965 mm. In one embodiment, the polyethylene tubing is melted, without burning it, in a small flame and pulled until it forms a small diameter tip, according to the demands of the user. The melted tubing is then cut into two, forming two tips 160 for two glass electrodes 135. Typically, a tip 160 formed in this manner has a diameter of about 10 μm to about 250 μm. Tips 160 with diameters in that range allow air-tight control of the area of the tissue being analyzed by allowing a portion 172 of the tissue 167 to be sucked into the tip 160 of the glass electrode 135.

Embodiments that include a glass electrode 135 for recording or stimulating tissue also generally include at least one structure that can function to transmit an electrical signal. In one embodiment, depicted in FIG. 6B, there are two structures that can function to transmit an electrical signal, a first wire 170 and a second wire 171. The structure that functions to transmit an electrical signal can be made of any conducting material, such as a conducting metal. Examples of conducting metals that can be used include but are not limited to copper, tungsten, silver, or an alloy thereof. In one embodiment, the structure that functions to transmit an electrical signal is made of silver which is coated with Teflon® in order to ensure electrical insulation. The structure that functions to transmit an electrical signal can include at least one wire. One exemplary configuration of the embodiment depicted in FIG. 6B has a first wire 170 and a second wire 171 that are made of a bare silver wire with a diameter of about 0.003 inches (0.008 cm), which when coated with Teflon®, has a diameter of about 0.0055 inches (0.14 cm).

Recording or stimulation of the tissue is generally achieved by passing a current either from the first wire 170 to the second wire 171 or from the second wire 171 to the first wire 170. This is most effectively accomplished when a portion 172 of the tissue 167 is sucked in at the tip 160, as depicted in FIG. 6C. In general, the tighter the suction, the better results are obtained for either recording or stimulating. The Teflon® coating is removed only at the tips of the first wire 170 and the second wire 171 so that they are not electrically shorted by contact at other portions along the first wire 170 or the second wire 171. The glass electrode 135 can also be used in connection with the fluid 174 to make an air tight system that includes a portion 172 of the tissue 167 by having the tip 160 of the glass electrode 135 filled with the fluid 174 that bathes the tissue 167.

The tip 160 can be attached to the glass tube 155 by using a ring 165. Generally, the ring 165 can be made of plastic, or PVC. Use of the ring 165 allows easy and quick replacement of the tip 160 if different diameter tips 160 are to be used. Both the first wire 170 and the second wire 171, run through the inside of the glass tube 155 and exit from the glass tube 155 at the end of the glass tube 155 that is away from the tissue sample.

The first wire 170 and the second wire 171 are generally connected to an electrical signal processor 169. The electrical signal processor 169 can include a number of different things, including, but not limited to an amplifier, a stimulator, a filter, or a computer. Alternatively, more than one electrical signal processor 169 can be connected to the first wire 170, the second wire 171, or both. The connection of the electrical signal processor 169 is well within the ordinary skill of one skilled in the art having read this specification. In one embodiment miniature pins 168 can be used to attach the first wire 170 and/or the second wire 171 to the electrical signal processor 169.

In one embodiment, the glass tube 155 can also function to provide suction. In such an embodiment, the glass tube 155 can be connected to an elastic tube 173 which can be connected to a syringe 174 at the other end. The size of the syringe 174 depends at least in part on the scale of the experiments to be carried out with the device 100. In one embodiment, the syringe 174 has a volume of about 1 ml to about 10 ml. The elastic tube 173 can be made of elastic material, as is known to those of skill in the art having read this specification. In one embodiment the elastic tube 173 is made of Tygon®. The elastic tube 173 can alternatively be connected to the glass tube 155 with a second ring 166. A T-junction 176 can also be configured with the syringe 174 to allow the internal negative pressure within the glass tube 155 to be “locked” or maintained.

Another embodiment of the invention utilizes a tip 160 that has the end that is in contact with the sample bent (for example by using a small flame) in order to allow the experimenter to rotate it along its axis, enabling more precise control of the tip 160 of the glass electrode 135.

In another embodiment of the invention a commercially available intracellular electrode holder with motorized micromanipulators can also be used with a device 100 of the invention. Examples of such commercially available devices that can be used with a device 100 of the invention include, but are not limited to, those commercially available from Narishige International USA Inc. (1710 Hempstead Turnpike, East Meadow, N.Y. 11554, USA), Siskiyou Design Instruments (S-D Instr. 110 S.W. Booth Street, Grant Pass, Oreg. 97526, USA), Axon Instruments (3280 Whipple Road, Union City, Calif. 94587, USA), World Precision Instruments (WPI, 175 Sarasota Center Boulevard, Sarasota, Fla. 34240, USA) and Luigs-Neumann (Feinmechanik und Electrotechnik GmbH, Boschsraβe 19, 40880 Ratingen, GERMANY). In one embodiment, the commercially available electrode is made by S-D Inc.

### WORKING EXAMPLES

The following examples provide a non-limiting illustration of the apparatus and methods of the invention.

### Example 1

### Exemplary Configuration of an In Vitro Chamber

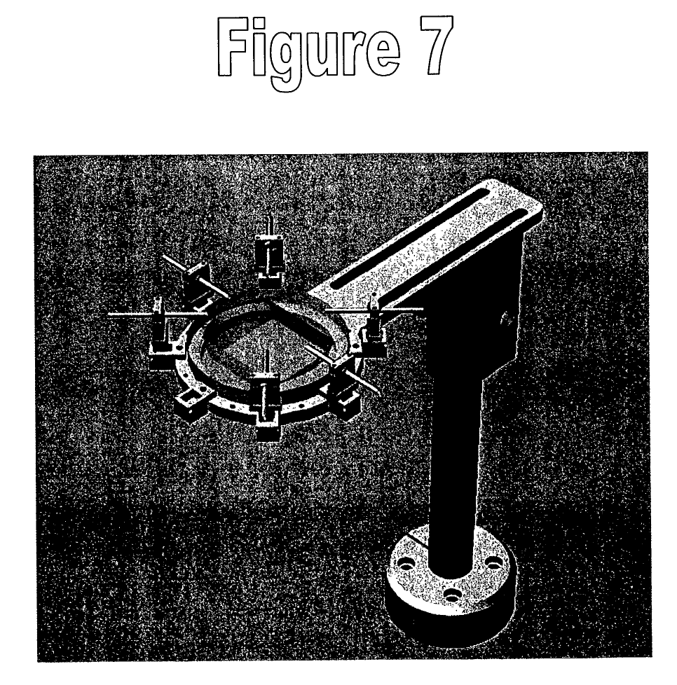

FIG. 7 depicts one specific example of a device in accordance with the invention.

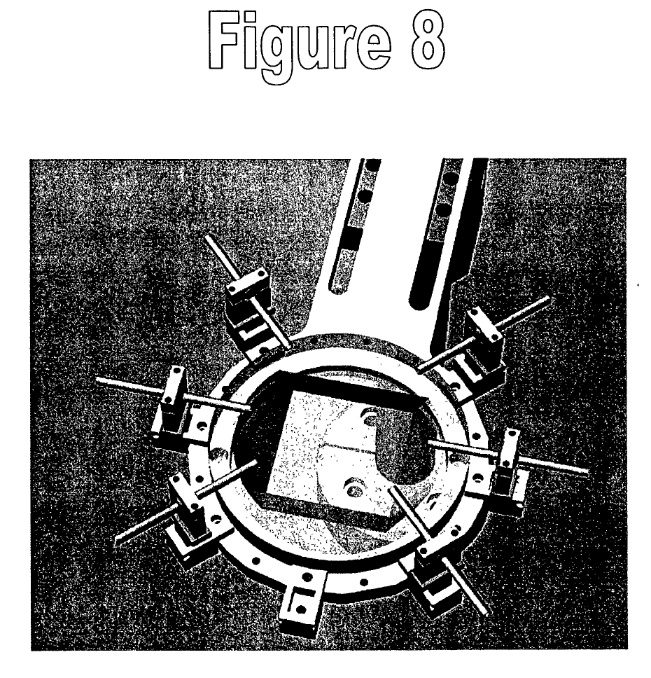

The chamber is made of Plexiglas® and was designed and manufactured using SmartCam Advanced Production Milling engineering software (EDS, Plano Tex.). The chamber was milled using a FADAL® automated mill (Fadal Engineering, Inc., Chatsworth, Calif.). The chamber included three main compartments: an inlet area, a specimen area and an outlet area. FIG. 8 shows a detailed view of the chamber, and indicates the three areas of the compartment. In this embodiment, the three compartments are connected by means of small holes (not shown in FIG. 8).

The chamber was attached to the base with a tight fit using a step-like arrangement. The chamber was equipped with a metal ring around the step-like circular edge in order to ensure that there was no free rotation of the chamber by means of small magnets found on the base.

The base was made of aluminum and was designed and milled using the same software and equipment as was the chamber. The base is a platform for the chamber and was mounted on the opposite side of a vertical metal bar which holds it in place on the anti-vibration station. The use of the vertical bar-aluminum base combination made the device easily integrated into a standard anti-vibration station with threaded holes on the station, as are commonly used in electrophysiological experiments. The base also had sixteen (16) threaded holes for attaching micro-manipulators thereto via the attachment assemblies of the micromanipulators.

The base had a hole at one end that allows the chamber to be positioned and supported throughout its periphery. This also allowed the chamber to be rotated as necessary. The chamber was tightly fitted to the base so that lateral movements were not allowed, but rotation around the z-axis was allowed. Such a combination of allowable movements allowed the user to adjust the specimen at various rotated positions during the course of an experiment without having to reposition the specimen. The chamber could be locked at different positions within the base by use of a metal ring plate that is fitted in the area where the chamber “seats” on the base and two magnets which are located in the base. The magnets allowed for movement through a slight manual effort by the user, but did not allow free movement of the chamber within the base.

This device also included a set of eight (8) micro-manipulators. Each of the micro-manipulators included: an attachment assembly, and a motion assembly. In this device, the attachment assembly of each micromanipulator was attached to the base with a small screw, and the motion assembly was attached to the attachment assembly with a small screw. The motion assembly included a ball assembly that included a ball, and a tube, and a ball housing that included an upper portion and a lower portion. The ball assembly was assembled by drilling a hole through the ball, inserting the tube through it and soldering the tube within the ball or tightening it with a mini-screw. The upper portion and the lower portion were then positioned around the ball as seen in FIG. 3B. The upper portion and the lower portion fit tightly enough around the ball so that the ball assembly could not fall out, but loose enough so that the ball assembly could not move within the space. This fit was accomplished by the two small screws that hold the upper portion and the lower portion together around the ball assembly.

The inlet area has fine holes (about 1 mm) which allow the artificial cerebrospinal fluid (ACSF) to be collected and slowly diffused to the specimen area. The bottom surface of the specimen area was coated with Sylgard® (Dow Corning, Silicone Elastomer, Sylgard 184). Sylgard® is a transparent elastic that is non-toxic to the tissue and enables the user to pin the tissue securely by means of fine micro-pins (0.1 to 0.2 mm pins) as well as allowing transmitted light from the microscope to illuminate the tissue under investigation. The outlet area was also equipped with fine holes so that the ACSF could be drawn out of the specimen area and into the fluid modulator where the ACSF had its temperature and oxygenation level monitored and regulated. The ACSF was stored in a glass container. The glass container was immersed and surrounded by distilled water in a larger container. A circulating generator controlled the temperature of the distilled water by means of a thermostat. In that manner, the ACSF could be warmed up to a more physiological temperature (i.e. 30° C.) when compared to room temperature (typically range: 20-25° C.).

The ACSF was fed into the chamber by gravity. The outlet was connected to a mini-pump, which in turn extracts the ACSF from the chamber and returned it to the ACSF reservoir. In the reservoir, the ACSF was oxygenated (bubbling with 95% oxygen and 5% carbon dioxide) continuously. The tubing used from the reservoir to the chamber (inlet) and from the chamber back to the reservoir (outlet) is any inert type of plastic tubing such as Tygon® or PVC.

Within the tube of the ball assembly was positioned a glass electrode. The glass electrode was fit within the tube so that it could be moved both in and out of the tube. The glass electrode was constructed as discussed above, using a glass tube with an outer diameter of 2 mm, a tip of polyethylene tubing with an inner diameter of 0.58 mm, and an outer diameter of 0.965 mm. The glass electrode has two wires with a bare silver wire with a diameter of 0.003 inches which is coated with Teflon® to a final diameter of 0.0055 inches.

This type of glass electrode can be used for DC or AC differential recordings. The potential (voltage) generated between the two wires of the glass electrode is amplified and converted (analogue-to-digital conversion) and stored in a computer for off-line analysis. The tighter the suction of the tissue at the tip of the electrode, the better the quality of the recording (i.e. electrical insulation between the two wires). If the tissue is not sucked in properly, ions will “bypass” the tissue and will “flow” from one wire to another which would effectively introduce an artifact or even eliminate the signal.

### Example 2

### In Vitro Extracellular Measurement on a Spinal Cord

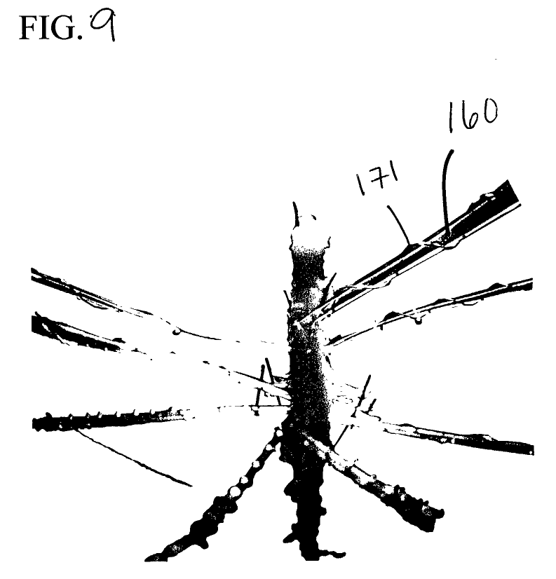

As an example of an actual use of a device of the invention, the device discussed in Example 1 was used to conduct experiments on a mouse spinal cord, see FIG. 9.

A spinal cord from a three day old mouse (Sp. mus musculus) was positioned in the tissue chamber and pinned down on the bottom surface of the chamber. The device had artificial cerebrospinal fluid (ACSF) circulated throughout the chamber. ACSF is a solution containing all the important ions required for neurophysiological studies. It does not contain all the substances that are found in the cerebrospinal fluid of the in vivo animal (such as proteins, enzymes, antibodies) but does have all the important ions. A typical composition of ACSF includes: NaCl (128 mM), KCl (4 mM), NaH₂PO₄*H₂O (0.58 mM), NaHCO₃ (21 mM), D-Glucose (30 mM), CaCl₂*2H₂O (1.5 mM) and MgSO₄*7H₂O (1 mM).

The eight (8) glass electrodes of the system were positioned in various areas on the tissue by fine manual movements of the micro-manipulators specific and accurate placement of electrodes in various parts of the tissue is fundamental to the success of the experiment. In the central nervous system (of which the spinal cord is a portion), different areas contain different types of neurons and/or fibers. Analysis of these different areas is the essence of most experiments on the central nervous system. With extracellular electrodes, such as the glass electrodes used in devices of the invention, the experimenter is able to study the response from neurons of a specific origin and physiological function. The electrodes were bipolar suction electrodes (the outer silver wire can be seen on the electrodes, as explained in detailed in FIGS. 6B and 6C) as explained above. FIG. 9 shows the spinal cord with the electrodes positioned thereon. As can be seen in FIG. 9, several of the ventral roots and/or dorsal roots, as well as other areas of the tissue were sucked into the tips of the electrodes, which resulted in a tight seal. The electrodes either provided stimulation, or were used to record signals.

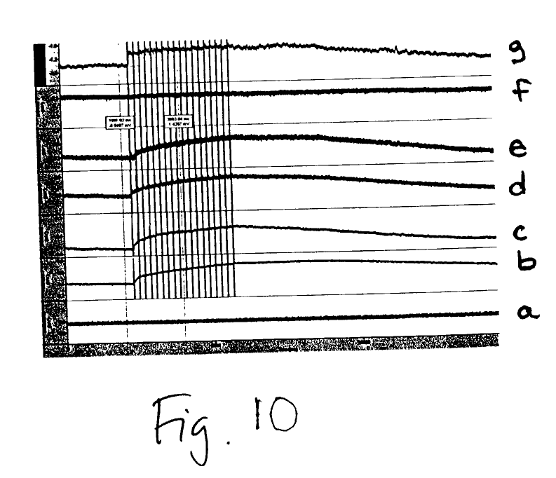

FIG. 10 shows extracellular recordings that were recorded using the device as described and configured above. The top six traces (b-g) are simultaneous recordings from six electrodes placed in the spinal cord in vitro chamber. The bottom trace (a) is from an electrode that was used to stimulate a specific area of the spinal cord.

The top six traces (b-g) are DC recordings (voltage without any filtering) from the different parts of the spinal cord. The traces reveal that a glass electrode, used in a device of the invention, can be utilized to record responses from a portion of the central nervous system. More specifically, the top six traces reveal that at the beginning, the baseline trace is followed by high frequency stimulation artifacts where it can be seen that the voltage was increased indicating that the tissue under each electrode was physiologically responding. Following the high frequency stimulation (from the electrode shown in trace a; since it was used to stimulate, it did not record anything, hence a flat baseline) all six electrodes indicate a response to a varying degree.

### Example 3

### In Vitro Extracellular and Intracellular Measurements on a Spinal Cord

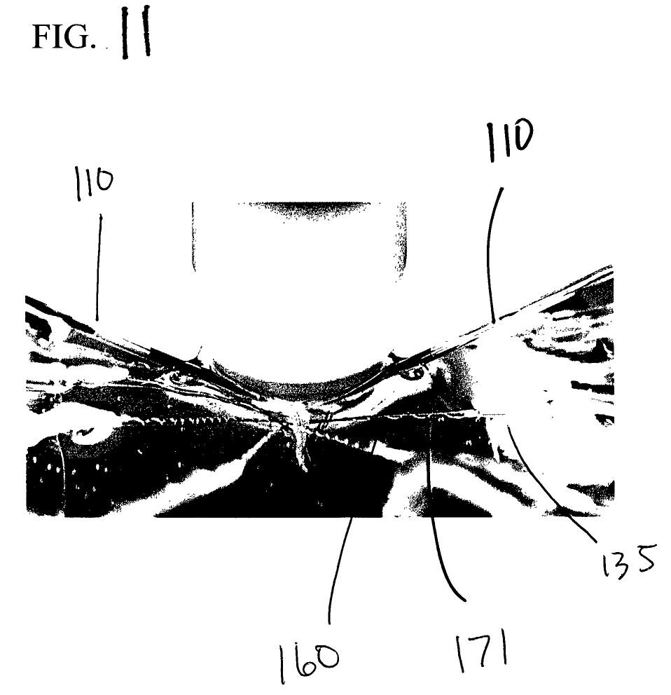

FIG. 11 shows a close up view of the in vitro chamber under a water-immersion objective (10×) of an epifluorescent microscope (Zeiss, Axioscope 1). The configuration used in this experiment was the same as that of Example 2 except that two intracellular electrodes 110 were also included. The micromanipulators were used to place the extracellular electrodes in the tissue in order to gain data and/or information from various populations of neurons within the spinal cord. Often, the response from a single cell (i.e. neuron) is required as well. For this reason, a different type of electrode was employed to achieve this.

In this case, glass electrodes filled with a slightly different physiological solution (in order to mimic the contents of the cell) were used in order to impale the cell and record either the voltage or the current across the cell's membrane. For this reason, a more precise microscope was employed in order to visualize the intracellular electrode with respect to the tissue. The intracellular electrodes were mounted on motorized micromanipulators since the cellular space is substantially much smaller and not visible to the naked eye (i.e. typical examples of cell diameters range from about 10 μm to about 40 μm). In the device used in this example, two intracellular electrodes can be easily applied on “top” of the extracellular electrodes approaching the tissue from a sharper angle. This can be important if simultaneous responses from populations of neurons (extracellular recordings) and from individual cells/neurons (intracellular recordings) are required.

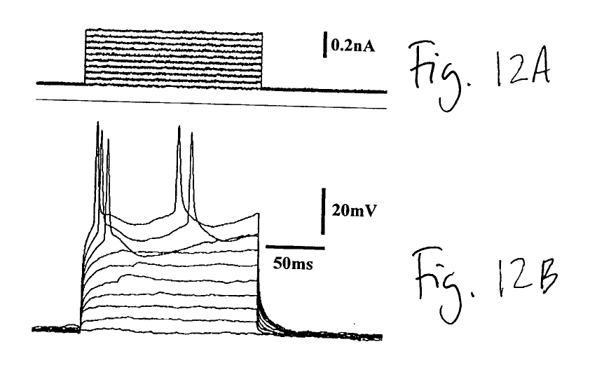

FIGS. 12A and 12B show current to voltage (I/V plot) relationships from a motorneuron obtained using the whole-cell configuration (current clamp). The bottom trace on each Figure signifies the baseline. FIG. 12A shows the current injected (depolarizing) and the traces in FIG. 12B show the resulting changes in membrane potential. Note that at the rheobasic current strength, the cell elicits orthodromically-evoked action potentials.

These traces show that the device of the invention can be used in combination with intracellular electrodes to record simultaneous responses from populations of neurons and individual cells or neurons.

The above specification, examples and data provide a complete description of the manufacture and use of the composition of the invention. Since many embodiments of the invention can be made without departing from the spirit and scope of the invention, the invention resides in the claims hereinafter appended.

The claimed invention is:

1. A micromanipulator comprising:
a ball housing having a space therein; and
a ball assembly, comprising a ball and a tube inserted through said ball;
wherein said ball assembly is movably positioned within said space in said ball housing, and said tube is securely positioned within said ball, wherein said ball imparts three dimensional movement to said tube through rotational movement of said ball within said ball housing.

2. The micromanipulator of claim 1, wherein said ball housing further comprises an upper portion and a lower portion that are configured around said ball assembly.

3. The micromanipulator of claim 2, wherein said upper portion and said lower portion are held tightly together by screws.

4. The micromanipulator of claim 1, wherein said ball assembly is one continuous component.

5. The micromanipulator of claim 1, wherein said ball and tube of said ball assembly are formed separately.

6. The micromanipulator of claim 5, wherein said tube is sealed within said hole in said ball.

7. The micromanipulator of claim 6, wherein said sealing is accomplished with soldering.

8. The micromanipulator of claim 1, wherein said ball has a radius of from about 3 mm to about 10 mm.

9. The micromanipulator of claim 8, wherein said ball has a radius of about 7 mm.

10. The micromanipulator of claim 1, wherein said ball is made of brass.

11. The micromanipulator of claim 1, wherein said tube has a length that is larger than its radius.

12. The micromanipulator of claim 1, wherein said tube has a radius of about 1 mm to about 2 mm.

13. The micromanipulator of claim 12, wherein said tube has length of about 1 cm to about 5 cm.

14. The micromanipulator of claim 1, wherein said tube is made of stainless steel.

15. The micromanipulator of claim 1, further comprising at least one attachment assembly.

16. The micromanipulator of claim 1, wherein said micromanipulator is used for manipulation of at least one analysis tool.

17. The micromanipulator of claim 16, wherein said analysis tool is chosen from the group consisting of: microelectrodes, suction tools, temperature probes, and holders for multi-barrel probes.

18. A device comprising:
(a) at least one micromanipulator comprising:
(i) a ball housing having a space therein;
(ii) a ball assembly, comprising a ball and a tube; and
(iii) an attachment assembly
wherein said ball assembly is movably positioned within said space in said ball housing, and said tube is securely positioned within said ball, wherein said ball imparts three dimensional movement to said tube through rotational movement of said ball within said ball housing;
(b) a chamber that functions to contain at least one sample;
(c) a base that functions to securely hold said chamber; and
(c) at least one analysis tool,
wherein said micromanipulator is reversibly attached to said base and said micromanipulator functions to manipulate said at least one analysis tool with respect to said sample in said chamber.

19. The device of claim 18, wherein said ball housing further comprises an upper portion and a lower portion that are configured around said ball assembly.

20. The device of claim 19, wherein said upper portion and said lower portion are held tightly together by screws.

21. The device of claim 18, wherein said ball and tube of said ball assembly are formed separately.

22. The device of claim 21, wherein said tube is sealed within said hole in said ball.

23. The device of claim 22, wherein said sealing is accomplished with soldering.

24. The device of claim 18, wherein said ball has a radius of from about 3 mm to about 10 mm.

25. The device of claim 18, wherein said ball is made of brass.

26. The device of claim 18, wherein said tube has a radius of about 1 mm to about 2 mm.

27. The device of claim 26, wherein said tube has length of about 1 cm to about 5 cm.

28. The device of claim 18, wherein said tube is made of stainless steel.

29. The device of claim 18, wherein said chamber further comprises an inlet area, a tissue area, and an outlet area.

30. The device of claim 29, further comprising a fluid modulator.

31. The device of claim 30, wherein said fluid modulator functions to circulate fluid through said inlet area, to said tissue area, to said outlet area, to said fluid modulator and back to said inlet area.

32. The device of claim 18, wherein said chamber is made of Plexiglas®.

33. The device of claim 18, wherein said analysis tool is chosen from the group consisting of: microelectrodes, suction tools, temperature probes, and holders for multi-barrel probes.

34. The device of claim 33, wherein said microelectrode is a glass electrode.

35. The device of claim 34, wherein said glass electrode comprises a glass tube, a tip, and at least one wire.

36. The device of claim 35, wherein said micromanipulator is configured to house said glass tube of said extracellular electrode within said tube of said ball assembly.
